(19) United States
(12) Patent Application Publication
(10) Pub. No.: US 2023/0074512 A1
(43) Pub. Date: Mar. 9, 2023

(54) SYSTEM FOR IMPROVING CENTRAL LINE HYGIENE

(71) Applicant: Vascular SafeGuard LLC, Wayland, MA (US)

(72) Inventors: Katya BALSIGER, Tualatin, OR (US); Ron BOR, Weymouth, MA (US); Claudia HOYEN, Marblehead, OH (US); Donald P. LOMBARDI, Boston, MA (US); Christopher H. REYNOLDS, Wayland, MA (US); Debra RIDLING, Woodinville, WA (US); Megan STIMPSON, Shoreline, WA (US); David A. VOGEL, Scottsdale, AZ (US)

(73) Assignee: Vascular SafeGuard LLC, Wayland, MA (US)

(21) Appl. No.: 17/941,462

(22) Filed: Sep. 9, 2022

Related U.S. Application Data
(60) Provisional application No. 63/242,118, filed on Sep. 9, 2021.

(51) Int. Cl. A61L 2/10; A61M 39/08

(52) U.S. Cl. CPC A61L 2/10; A61M 39/08; A61M 2039/087; A61L 2202/15; A61L 2202/24; A61L 2202/122

(57) ABSTRACT

A system for improving central line hygiene, including a chamber for containing central line hubs that protects the central line hubs from germs, and optionally including a wand for irradiating the chamber and measuring parameters of the chamber. A kit including the chamber and wand of the system, and instructions for use. A method of protecting a central line hub from germs by enclosing the central line hub with the chamber, and preventing germs from entering the chamber.

### BACKGROUND OF THE INVENTION

### 1. Technical Field

The present invention relates to compositions and methods for providing safe and efficient vascular access. More specifically, the present invention relates to compositions and methods for disinfecting, blocking germ access, and preventing infection in central lines.

### 2. Background Art

Vascular access can be obtained by intravenous lines (IVs) or by central lines (CLs). Both of these arrangements connect external tubing to the circulatory system of humans or other animals. Central lines provide the additional feature of one or more internal tubes, or lumens, that proceed inside a vessel, generally a vein, from the access point to a location near the superior vena cava of the heart. The principal benefits of central lines are faster and more predictable dissemination of the fluids introduced, and the ability to introduce powerful or transient medicines that might degrade or prove toxic if introduced into the narrow and slow vessels of the extremities. This ability to convey fluids directly to the center of the circulatory system gives central lines their name. Because central lines provide immediate access to large vessels with rapid flow rates, they enjoy a reputation for practical dependability in critical care. For the same reasons, central lines are also at risk for introducing life-threatening infections.

Typical access points for IVs include the veins of the forearm and the back of the hand. Typical access points for central lines include the femoral, jugular, and subclavian veins. In the special case of a PICC line, described below, a central line can gain access via veins in the elbow and forearm area. At the access point, the external segment of the central line emerges from the skin. In some medical communities, this external portion is termed the central line tail or just the tail. The external segment or tail is generally 5 to 18 inches in length and ends with a hub. The hub is typically a multipurpose fitting that facilitates connection of the tail to other modular elements in the overall tube set. Representative hub configurations include terminated-mode and through-mode. In terminated mode, the tail is sealed off via a cap when the line is not in use. In through-mode, the hub fitting joins the tail to another tube or fitting, e.g., to connect to a blood bag, drug infusion pump, manifold, or various fittings through which medicine may be injected and from which blood may be drawn. Diaphragms and specialized fittings exist to permit both needle-based and needleless access.

There are various forms of central lines with various names. Examples include Central Venous Line or Catheter (CVL, CVC), typically inserted in the neck, chest, or groin, or “PICC” lines, Peripherally Inserted Central Catheter, typically inserted in the elbow-hand region. CLs are also referred to by nicknames. The most prominent product nickname is the Hickman, after Seattle's Robert O. Hickman, M. D., inventor of a transformative catheter. Initially developed for cancer patients, the Hickman catheter has grown into widespread use. Other shorthands include Broviac, Groshong, and Powerline, each with special features. Venous Ports, implanted under the skin, require needle access and do not have conventional hubs. In all cases except venous ports, the central line catheter exits the skin opening and forms an external segment or “tail” outside of the body that terminates with a hub. FIG. 1 shows a central line extension with the major components of the exterior portion of a central line ending with a single hub. In practice, the external portion can include a manifold from which several hubs ensue. At many hospitals, plural hubs are termed fuses, and three or four can collectively be termed a tri-fuse or quad-fuse. FIG. 2 shows a quad-fuse with backflow check valves and pinch clamps.

While central lines are very effective tools, they require meticulous installation and maintenance for safe operation. It is imperative that hubs be kept clean. Achieving proper hygiene is difficult due to widely varying conditions that complicate procedures, as well as fittings with nooks and crannies that can harbor germs.

Some central lines are in place for 48 hours in response to emergencies; others are in place for years for long-term therapies and chronic conditions. These widely varying contexts can stymie attempts to develop a single, consistent hygiene maintenance regimen for all conditions. This observation reveals why central line hygiene protocols vary between hospitals, even between hospitals recognized for best practices.

Hub connections provide recesses, gaps, and voids that can protect germs while they reproduce and form colonies. Advances in the study of infections indicate these colonies can be more harmful than the increase in the quantity of germs suggests by itself. The work of Dr. Lori L. Burrows of McMaster University reveals that colonies of germs may form biofilms characterized by rugged layers of interlocking proteins. These biofilms can be highly resistant to scrubbing and solvents. Some biofilms appear to be protected from antimicrobial treatment by layers of dead cells that serve as a defensive wall for the living cells beneath them. Some studies suggest that scrubbing the biofilm can pierce the top dead layer and reveal the living cells underneath, possibly worsening the risk of infection. Biofilms on hub fittings can provide a beachhead from which germs enter the central line and then the bloodstream. Moreover, the fittings are sometimes pre-loaded with germs because of their location. In young patients the best access point may be a femoral vein due to its preferred size. This location, discussed more below, is in the diaper area where hubs quickly become coated with feces, urine, and attendant germs.

Blood provides germs with mobility and a warm, nutrient-rich environment. Germs that trespass into a central line can quickly grow into a widespread, life-threatening infection. Such an infection is called a Central-Line Associated Bloodstream Infection, or CLABSI. Numerous studies demonstrate that CLABSIs are preventable, and much progress has been made to reduce the incidence and severity of CLABSIS in the past decade. Watershed studies and initiatives include the Michigan “Keystone Study” and the On the Cusp: Stop BSI program coordinated by the Agency for Healthcare Research and Quality, among many others. The key steps to reducing vascular-access infections have been celebrated for their practicality. The breakthrough work of Dr. Peter J. Pronovost, et al., for example, highlights succinct decision trees and short, practical checklists to avoid installing lines that will be underused, to regularly monitor lines and question whether they continue to be necessary, to remove unneeded lines promptly, and to maintain central line hygiene. It is now best practice in U.S. hospitals to conduct “Scrub the Hub” campaigns to train staff in ICUs and some wards. These campaigns, like handwashing campaigns, feature diligent scrubbing for mandated periods conducted at frequent intervals. To urge compliance, the largest payers have enacted policies under which CLABSIs are designated a nosocomial or “hospital-acquired” condition (HAC) for which treatment will no longer be reimbursed. By act of Congress, CMMS, the payment and compliance arm of Medicare and Medicaid, is not permitted to reimburse for CLABSIs in adult patients, except in rare cases. Consequently, hospitals must pay the costs of mitigating CLABSIs on their own. CLABSIS are presently the first- or second-most costly HAC in the U.S. healthcare system. The CDC and the Association for Vascular Access have estimated >250,000 CLABSI cases in the U.S. per year, a morbidity rate of 20 percent reflecting 50,000 to 65,000 deaths, and a national economic cost of >$1 billion. The Institute for Pediatric Innovation (IPI) and some of your inventors have interviewed clinicians and hospital executives at scores of medical centers in the US. These interviews reveal widespread belief that the actual costs of CLABSIs in lives and dollars are likely to be higher than the figures above. This inference follows because nearly every CLABSI is a comorbidity along with the principal diagnosis that caused a patient to be admitted to the hospital in the first place. Death certificates and related reports may cite the principal or sometimes the primary diagnosis, not secondary diagnoses such as hospital-acquired conditions.

In the U.S. from approximately 2006 to 2015, hospitals focused intensely on implementing practical techniques such as the Pronovost checklists. These steps succeeded in reducing CLABSI rates sharply. By 2016, however, rates had plateaued. The 2017-2020 period provided increasing evidence that most easily enacted steps had been implemented and the field of CLABSI reduction had reached a point of diminishing returns. Currently, CLABSI rates remain unacceptably high, and further reductions using existing tools will require unacceptable amounts of time, money, or both. As with hand washing, scrubbing a central line hub for twice as long will not on its own reduce the number of colony-forming units of a certain germ by half. Moreover, clinicians and nurses do not have time to improve hygiene by performing existing protocols with existing tools more frequently or for longer periods. Many action plans and safety campaigns promise improved outcomes and fewer complications in exchange for impractical workload increases, compliance surveys, and additional training. After reviewing the declining results of certain timeworn training programs, some hospital executives have intimated that urging providers to apply the same tools more diligently to combat CLABSIs is a tired strategy that hurts morale and contributes to burnout. Worse, several hospitals have shared with the inventors that institutional stress may increase CLABSI rates. Two top-tier hospitals have disclosed that during the COVID pandemic of 2020, CLABSI rates increased even though a biological connection between these two conditions has not been identified. Continued progress on CLABSIs requires new tools that reduce CLABSI rates while imposing less training time and less implementation time on overworked staff than current tools.

Data from the Center for Disease Control's National Healthcare Safety Network (NHSN), along with confirming sources, associate the following pathogens with CLABSIS: coagulase-negative Staphylococci (e.g., S. epidermidis), Enterococci, Staphylococcus aureus, Klebsiella, Enterobacter, Pseudomonas, E. coli, Acinetobacter, and Candida species. Anti-microbial agents such as chlorhexidine gluconate, isopropyl alcohol, povidone-iodine, and silver can dramatically reduce the growth rates of these germs. These anti-microbial agents are effective only for short periods. For example, alcohol is frequently positioned to kill germs for just a few minutes before it evaporates, after which efficacy declines. Likewise, other anti-microbial agents can rinse away during daily routines such as showering. Current dressings and fittings struggle with the trade-offs between patient comfort and effective protection.

The central line provides a direct channel from the access point to the upper chest. Consequently, access points into large vessels that can accommodate penetration and the course of the lumen(s) without damage are preferred. In certain patients, notably infants, this preference leads to placement of the central line in a femoral vein, even in the presence of diapers. This is a widespread, time-honored industry practice due to the wide vein.

Currently in the U.S., the average CLABSI rate is approximately 0.8 cases per thousand central-line days. This rate corresponds to approximately 140 new CLABSI cases per day in the US. In industrialized nations outside the U.S., the rate averages four times higher. CLABSIs are represented in the International Classification of Diseases ICD-10 schedule. The disease category is T80, with various suffixes for type of catheter (Hickman, PICC, triple-lumen, etc.).

In sum, the deaths and costs associated with CLABSIs, combined with manifest preventability, the threat of non-reimbursement, staff burnout, and public ranking of outcomes by major payers such as CMMS, altogether present an industry mandate.

Therefore, there remains a need for an easy-to-use, robust system to promote central line hygiene.

### SUMMARY OF THE INVENTION

The present invention provides for a system for improving central line hygiene, including a chamber for containing central line hubs that protects the central line hubs from germs, and optionally including a wand for irradiating the chamber and measuring parameters of the chamber.

The present invention provides a kit including the chamber and wand of the system, and instructions for use.

The present invention provides for a method of protecting a central line hub from germs by enclosing the central line hub with the chamber and preventing germs from entering the chamber.

### DESCRIPTION OF THE DRAWINGS

Other advantages of the present invention are readily appreciated as the same becomes better understood by reference to the following detailed description when considered in connection with the accompanying drawings wherein:

FIG. 1 is a top view of a typical configuration of the external elements of the central line and is labeled prior art;

FIG. 2 is a top view of a quad-fuse central line labeled prior art;

FIG. 3A is a top perspective view of a chamber in an open position with a central line hub and tubing lines, and FIG. 3B is a top perspective view of a chamber in a closed position with a central line hub and tubing lines;

FIG. 4A is a top perspective view of a chamber that fits an IV Y-site, FIG. 4B is a top perspective view of a chamber that fits a hub with a trifuse, and FIG. 4C is a top perspective view of a chamber that fits a texium connector with a living hinge;

FIG. 5A is a top view of a chamber with a standard IV Y-site in through mode, and FIG. 5B is a top view of a chamber with a catheter with a maxzero cap in terminated mode;

FIG. 6A is a top perspective view of a chamber in an open position including sub-chambers, and FIG. 6B is a top perspective view of a chamber in a closed position; and

FIG. 7A is a top view of a chamber in an open position with a hub and tubing, and FIG. 7B is a top view of a chamber in a closed position with a hub and tubing.

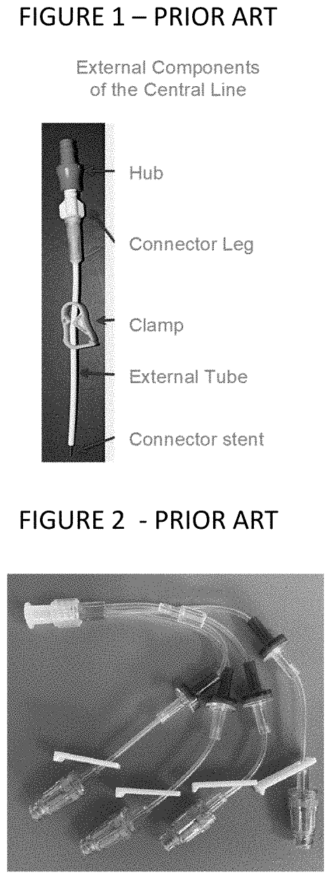

### DETAILED DESCRIPTION OF THE INVENTION

The present invention is directed to a system for improving central line hygiene, shown at 10 in the FIGURES, including a chamber 12 for containing central line hubs 14 and other related vascular-access fittings that protects the central line hubs 14 from germs. The system 10 can also include a companion tool (wand) 16 that engages with the chamber 12 to disinfect the inside of the chamber 12 and monitor operation.

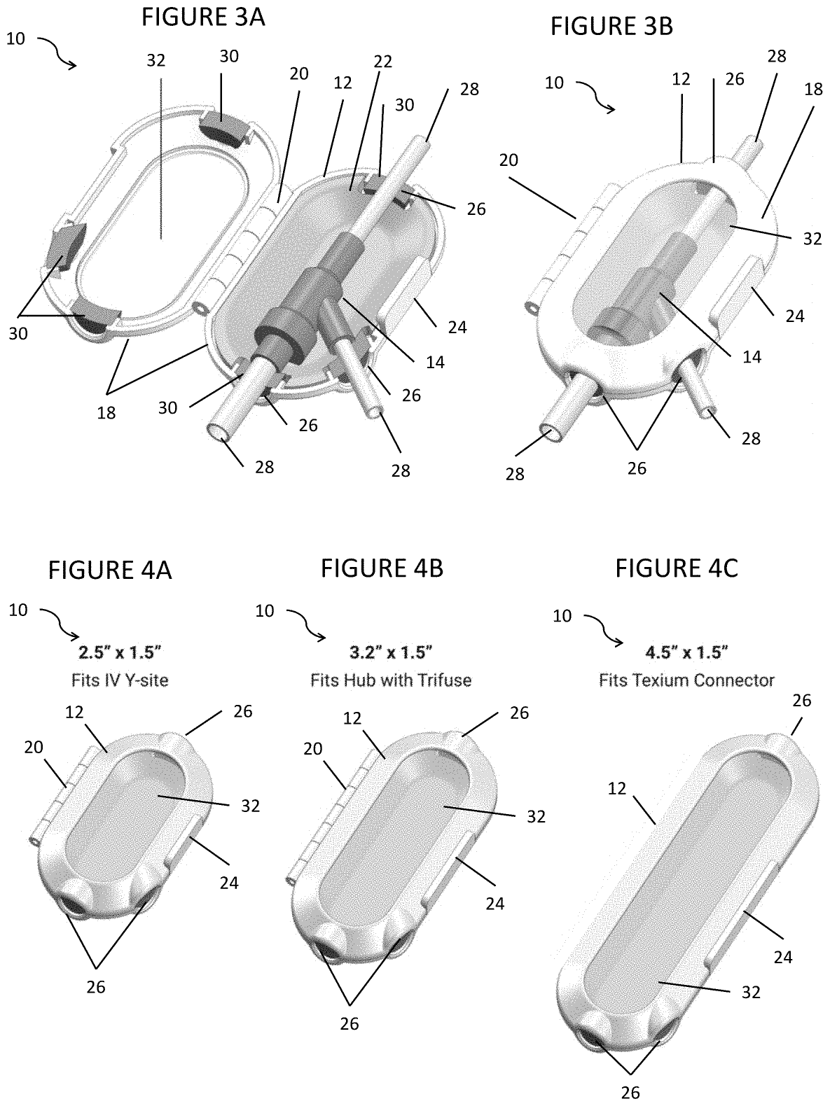

As shown in FIGS. 3A, 3B, 6A, 6B, 7A, and 7B, the chamber 12 includes two concave halves 18 forming a clamshell design. Each concave half 18 is operatively attached through a hinge 20 so that the concave halves 18 can rotate toward each other to form the chamber 12 that envelops the contents placed inside (central line hub 14), and so that the concave halves 18 can also rotate away from each other to open the chamber 12 and provide access thereto. The function of the chamber 12 is to provide a barrier to keep germs away from the central line hub 14 and vascular access fittings placed inside. The chamber 12 can be made in different sizes to accommodate different hubs and connectors, as shown in FIGS. 4A-4C.

The chamber 12 is made of a relatively stiff material, such as polyamide (e.g., generic nylon), polyoxymethylene (POM, sometimes known by the trademark DELRIN® (DuPont)), polypropylene, acrylonitrile butadiene styrene (ABS) such as ZYLAR® 960 (INEOS Styrolution), a naturally transparent amorphous thermoplastic such as polycarbonate, or a UV-transmissive acrylic. Its Young's Modulus, shear modulus, and geometry are engineered to support a clamping action to ensure effective sealing of the chamber 12. Sealing results from closing leverage provided by the hinge 20 acting as a fulcrum on one side of the chamber 12, a latch 24 on an opposite side of the chamber 12, and the chamber 12 itself between them.

The chamber 12 can also include a skin 22 with all or portions made of a relatively compliant material, such as, but not limited to, medical-grade ethylene vinyl acetate (EVA), silver impregnated foam, medical-grade or biocompatible silicon rubber, or a transparent thermoplastic capable of elongation such as Tanac CRYSTAL GEL® (more than 1000% elongation capabilities). The skin 22 provides the barrier against germs and contributes to the sealing action when compressed by the chamber 12. The skin 22 can be operatively attached to an outside of the chamber 12 (an exoskeleton), to an inside of the chamber 12 (endoskeleton), or to both. The skin 22 can include radiusing and chamfering to avoid protrusions and encourage reduction of pressure injuries to the patient's skin.

The chamber 12 can be formed by a single, extended mold operation with two or more injections of material. Sometimes called over-molding, this process can produce a coated chamber 12 or both the chamber 12 and the skin 22 in one molding cycle. The EVA or similar foam of the skin 22 can be heated and compressed in selected regions to remove cellular voids and stiffen the region, in effect fabricating the chamber 12 and the skin 22 in the same operation.

The chamber 12 includes a latch 24 that allows a user to maintain the concave halves 18 of the chamber 12 to remain in a closed position that effectively seals off the chamber 12 from its environment. The latch 24 includes a release mechanism with a combination of some or all of the following features. In single-use mode, the latch 24 engages and remains closed once; after the latch 24 is released by the release mechanism, the latch 24 does not re-close. In re-closable mode, the latch 24 engages, opens via the release mechanism, and subsequently latches again. This cycle of re-closability can be repeated indefinitely and can be useful to execute flushing protocols for proper maintenance of the central line. In a rapid-release mode, suitable for emergency removal or for convenience, a rapid release mechanism or “zip-strip” effects the immediate disintegration of the latch 24, the hinge 20, the chamber 12, or a combination thereof such that the chamber 12 falls away to provide rapid, unfettered access to its contents. The release mechanism can include a tether to gather the sundered pieces together after disintegration, thus reducing choking risk upon removal of the device. The tether can be formed from dedicated material or by dual purposing a portion of chamber 12 or skin 22 material. In a child-resistant or tamperproof mode, the release mechanism does not operate unless a procedure generally unknown by patients or requiring a tool generally unavailable to patients is performed. In practice, useful combinations of the modes above can be affected through the inclusion and exclusion of separate, individual elements, thus providing various embodiments. Alternatively, to improve manufacturing and simplify logistics, some elements can provide compound functionality or switchable functionality. For example, the latches 24 and release mechanisms for single-use and re-closable modes can result in different embodiments of the invention, or a single adaptable embodiment can allow the user to select the desired mode of operation in the field.

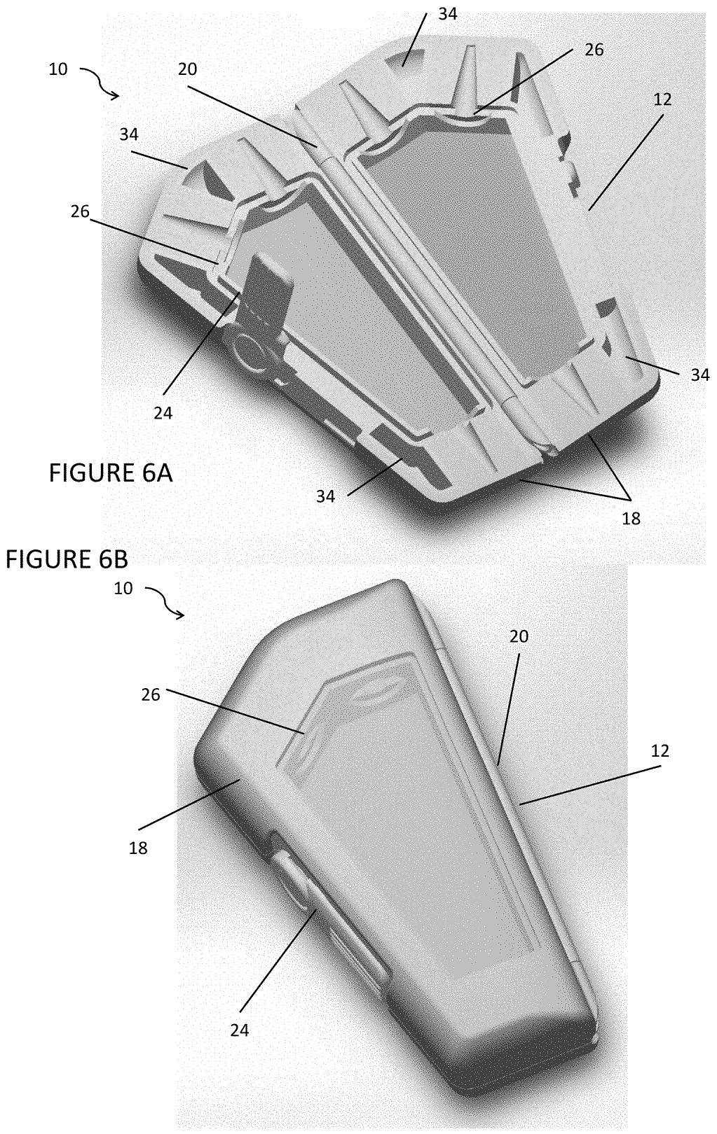

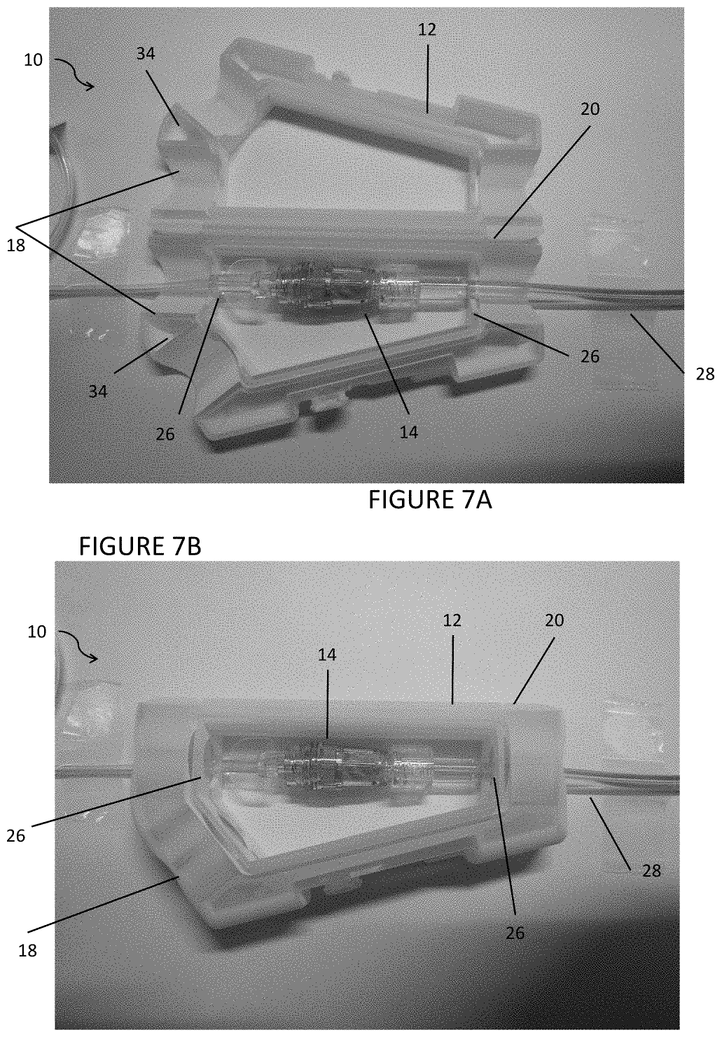

The hinge 20 can be formed by tunnels connected to the concave halves 18, such as in FIGS. 4A and 4B. The tunnels are coaxially aligned and secured by a hinge pin. Alternatively, ‘over’ and ‘under’ protrusions from a concave half 18 can align with, grasp, and linearly pivot along complementary protrusions on the other concave half 18 to effect a hinge. In a preferred embodiment, a living hinge 20 can be formed by an engineered thickness of durably flexible material, shown in FIG. 4C. The living hinge 20 obviates gaps along the interface of the tunnels and can provide superior sealing. In another preferred embodiment, surfaces of the skin 22 are positioned adjacent to the hinge 20 so that upon closure a continuous unbroken surface results to bar germs, even if the hinge 20 has gaps. Alternatively, a single surface of skin 22 can be folded and the fold line collocated with the hinge 20 axis to affect a seal even if the hinge 20 has gaps. With careful selection of the thickness of skin 22, the location of creases or folds, and engineered adhesives, the skin 22 can provide hinge action itself, as well as barrier action free of gaps. An adhesive can be added or can be engineered by selective melting of the skin 22 material to affect a localized adhesive. For example, when the skin 22 is comprised of ethylene vinyl acetate material with a vinyl acetate-to-ethylene ratio of approximately 12% by weight, the material can perform as a hot-melt adhesive. The hinge 20 can include sensor and associated electronics to detect and record the opening and closing of the chamber 12 in order to collect data useful for monitoring operations and obtaining reimbursement. This can be accomplished with a cavity adjacent the hinge 20, a ribbon of magneto-strictive material oriented in the cavity, and a protrusion about the hinge 20 axis that impinges upon or releases the ribbon, thus effecting an opening- and-closing sensor amenable to polling with passive electronics. The hinge 20 can also incorporate active electronics for embedded clocks and data logging. U.S. Pat. No. 7,882,732 discloses an apparatus for monitoring the pressurization in a tire that has a magneto-mechanical pressure sensor in or on the tire and an electromagnetic excitation system. Such an apparatus can be modified for use in the chamber 12 to avoid or limit ferromagnetic metal in the chamber 12. Any sensors in the system 10 can communicate with Internet-of-things (IOT) systems to report on chamber 12 readiness and utilization. Sensors can also be housed in a sub-chamber 34 located at any suitable location on the chamber 12 (shown in FIGS. 6A and 7A).

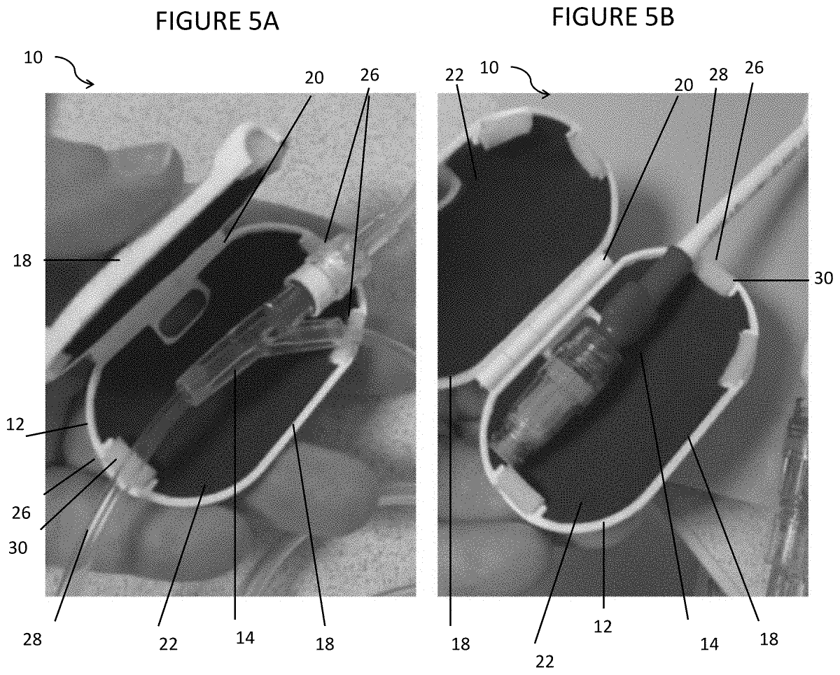

The chamber 12 includes at least one portal 26 through which at least one line of tubing 28 enters the chamber 12. When there is one portal 26 permitting one tube 28 entrance, the chamber 12 works in terminated mode to protect the end of the tube and the terminal fittings, if any (such as in FIG. 5B). When there are two suitably aligned portals 26, an external portion of the central line 14 can enter the chamber 12 through a first portal 26 and exit through a second portal 26, thus implementing through mode (such as in FIG. 5A), distinct from terminated mode, and providing protection for a hub 14 or fitting that connects a first section of tube to a second section. A third portal can provide further access, e.g., for blood draws, tests, or accessories. FIGS. 3A-3B show chamber 12 arrangements for three portals 26 to accommodate Lambda- or Y-fittings in regular use at some hospitals.

The portals 26 can be different sizes to accommodate various tubing set-ups. A simple arrangement of equal-sized portals 26 at noon and six o'clock can accommodate a typical central line 14 tail in through-mode with a basic connector. Alternatively, a large portal 26 and, for example, several smaller portals 26 can facilitate protection of a manifold hub fitting with one main entry line and an array of smaller fuses. The fuses can remain in the protection of the same chamber 12 as the manifold, or they can exit via portals 26 and receive protection through subsequent devices.

The portals 26 can be formed by opposing semi-circles set into each half of the concave half 18. Each semi-circle contains and positions a sealing mechanism 30 of a half-torus of silicon rubber, foam, or laminate of materials of appropriate compliance to encompass a seal with its opposing semi-circle upon closure around the tubing 28. A conical arrangement of a series of half-toruses of increasing radii can affect a sealing mechanism 30 that can accommodate a range of tubing diameters. The concave half 18 can include over-force limiters, extensions in each half of the concave half 18 which meet upon closure to limit the closing force upon the seals. In practice, the relationship between the stiffness of the chamber 12, the compliance of the skin 22 barrier, the compliance of the sealing mechanism 30, the clamping pressure or purchase of the latch 24, the slack of the hinge 20, and the size and compressibility of the tubing 28 are engineered to provide a sealing pressure that keeps potential contaminants out of the chamber 12 while maintaining standard flow in the tubing. The chamber 12 and sealing mechanism 30 can include actuators, pawls, teeth, or cusps that engage each other to form the sealing mechanism 30 around the tubing 28 to affect a controlled seal. The sealing mechanism 30 can include tethered plugs so they seal with or without tubing 28 inserted. These plugs can be removed by the healthcare provider as part of the installation workflow, or the seals and tethers can be arranged so that the insertion of tubing into a portal 26 causes separation of the sealing mechanism 30 and its plug. The sealing mechanism 30 can hold tubing 28 securely under routine pressure, while releasing its grip if yanked (graceful failure).

The sealing mechanism 30 can be affected by a low-durometer silicon rubber material positioned in the portal 26 so that it is pressed upon by the opposing sealing mechanism 30 in the opposing concave half 18 as the device is closed. The combination of this pressure and the highly compliant, low-durometer material effects a seal. The durometer or Shore value of the material is engineered to permit flow under pressure while ensuring that the material is not independently fluid. The amount of low-durometer silicon rubber material in the sealing mechanism 30 can be engineered to provide an effective seal when no tubing 28 is inserted, in which event the low-durometer silicon rubber itself fills the volume of the seal. The same material also provides an effective seal when tubing 28 within a range of acceptable diameters has been inserted, as the material is pressed throughout the volume of the sealing mechanism 30 and around the tubing 28. To accommodate the change in fill volume both without tubing 28 and with an acceptable range of tubing 28 diameters, any excess silicon rubber material can be shunted by a release conduit and overflow receptacle or by a flexible wall.

The interior of chamber 12 can be enhanced by desiccants for germ and humidity control, by anti-microbial agents, and by admixtures to facilitate storage, transport, and proper sealing across useful ranges of humidity, vibration, and temperature.

The low-durometer silicon rubber material can provide tack as well as sealing to facilitate insertion of the tubing 28 and help keep the tubing 28 in place prior to closure of the device.

The halves 18 of the chamber 12 need not operate identically. One half 18, for example, can be constructed of opaque foam as above, while a second half 18 can include an observation aperture 32 made of rigid or elongation-capable transparent material, either inserted into an opening in the skin 22 or as a complete replacement for the skin 22 in its half 18. The observation aperture 32 allows clinicians and patients to check for leaks from the enclosed fittings, leaks into the chamber 12 from outside, and other potential problems. It also allows new users of the system 10 to become familiar with and confident in its operation. A leak-responsive coating can be applied to a designated area on the inside of the chamber 12, and the observation aperture 32 can be oriented to facilitate viewing of this area (a semaphore mode). The coating changes color and/or shape upon contact with selected fluids. This enables monitoring of the chamber 12 and its contents for contamination. The observation aperture 32 can be composed of UV-transmissive material to work with the wand 16.

The chamber 12 can further include a recess or sub-chamber 34 adjacent to the hinge 20 or latch 24 that contains an anti-microbial or hygiene-enhancing agent in a capsule with frangible seals or burstable material collocated near a barb, pin, or similar structure that causes the capsule to open upon closure, thus releasing the agent into the chamber 12. This further provides protection of the central line hub 14 from germs. The chamber 12 can include grooves, runs, or wicking agents to aid dispersion of the released agent.

The interior of the chamber 12 can be nano-enabled to mitigate reproduction of germs and formation of biofilms. For example, the interior can be nano-textured to discourage germ attachment and growth. Nanotubes can be positioned on the interior of the chamber 12 to release preventative agents.

The chamber 12 can further include shoulders, wings, or extended flanges positioned around the perimeter to facilitate taping down or otherwise attaching the chamber 12 to the patient or an appropriate object such as a sling or diaper, thus stabilizing the central line exit tubing 28 without necessitating application of tape or adhesives to the tubing 28 itself or near the exit wound, which application can increase the risk of biofilm generation.

The wand 16 is a disinfection and data collection tool that optionally works with the chamber 12 to enhance the system 10. This enhancement addresses two needs. First, barrier-mode germ control can be a double-edged sword. Proper maintenance of the barrier is required; otherwise, there is risk that the barrier intended to keep germs out of a region may instead hold germs in and create an incubation chamber that fosters growth of germs and biofilms. Second, healthcare payers increasingly mandate that medical centers provide care on a pay-for-performance basis. In particular, payers are increasingly averse to paying for so-called hospital-acquired conditions, or HACs. This trend already includes the largest payers in the United States, and it is accelerating in both the number of payers/insurers involved and the types of care delivery included. Data measurement and logging are needed to ensure sound medical outcomes through protocol compliance and sound revenue recognition through prompt reimbursement. The means of data collection should fit established care-delivery workflows to avoid further burdening over-worked staff, or, ideally, the mode of data collection should gather data while also saving healthcare providers' time.

The wand 16 is a portable tool in a form suited to bedside care delivery. When placed adjacent to the observation aperture 32 of the chamber 12 (with the wand 16 being in the shape and size of a TV remote control or large cell phone), or, in some embodiments, when clamped over the entire chamber 12 to surround it (with the wand 16 being in the shape of an oversized clamshell chamber), the wand 16 irradiates the chamber 12 with ultra-violet (UV) light. Simultaneously, the wand 16 measures the date, time, frequency, intensity, and other relevant parameters of central line maintenance events.

The wand 16 can operate without an activation button and automatically recognizes a chamber 12 when held immediately adjacent to the observation aperture 32 or clamped around the entire chamber 12.

The wand 16 includes an array of UV LEDs, emitting at 260=λ nm, a region of wavelengths termed “UV-C.” Control circuitry can activate only those LEDs which are in direct contact with the observation aperture 32, thus preventing light leakage and potential risk to patients' and caregivers' eyes and skin. This can save care-givers time and enables a single wand 16 to perform with chamber 12 of various sizes and observation aperture 32 configurations. In a further embodiment, the chamber 12 includes UV-transmissive light guides whose shape and index of refraction convey light via total internal reflection from a wand-engaging port on the outside of the chamber 12 to and throughout the inside of the chamber 12. U.S. Pat. No. 4,257,084 discloses methods of projecting light that can be used herein. The entire chamber 12 can be made of UV-transmissive material to simplify fabrication. The chamber 12 can also be coated or semi-coated with a reflective material to improve dissemination of the light. Controls for the array of UV-LEDs in the wand 12 can include circuitry that monitors the active time of individual LEDs and activates unused LEDs to maintain overall light output as the LEDs age.

The wand 16 or the chamber 12 can include jigs that position tubing in desired orientations, facilitate set up and installation, and/or enable easy confirmation of tubing sizes and durometers appropriate for a specific chamber 12.

The chamber 12 can include a pigmented region that fluoresces upon exposure to the wand 16 to assure the user that it is operating, essentially showing a status of the chamber 12. The chamber 12 can include two regions that fluoresce, one that brightens fast and the other slowly; when they reach the same brightness level the disinfection cycle has been successfully completed. The chamber 12 or the wand 16 can include an electro- or photo-chromic gel that changes color to confirm a completed cycle.

The present invention provides a kit including the chamber 12 and wand 16 of the system 10, as well as instructions for use. Barcodes and/or QR codes can be printed on packaging or on the chamber 12 and wand 16 themselves for convenient access to training videos and instructional guides for both providers and patients. Mustering strips can be included as a bulk packaging component to keep several chambers 12 tidy and ready for use at the point of care to encourage prompt replacement.

The present invention provides for a method of protecting a central line hub 14 from germs by enclosing the central line hub 14 with the chamber 12 and preventing germs from entering the chamber 12. The chamber 12 can be effectively sealed from germs through the hinge 20 and latch 24. The skin 22 can further provide sealing action. When it is desired to open the chamber 12, the release mechanism of the latch 24 can be actuated. The method can further include collecting data regarding opening and closing of the chamber 12. The method can further include the step of checking for leaks in the chamber 12 through the observation aperture 32. The method can further include the step of irradiating the chamber 12 with UV light from the wand 16 to remove germs inside the chamber 12, and the chamber 12 can fluoresce once exposed to the wand 16. The wand 16 can also be used to measure parameters of central line maintenance events as described above.

The system 10 provides several advantages. It provides an intelligent chamber 12 that protects the central line hub 14 while also signaling that it is working. There is an intuitive workflow that facilitates ease of training and ease of use, functional comfort to avoid pressure injuries to the skin, and data logging capability to monitor status and support reimbursement. The chamber 12 is ruggedly protective yet also comfortable against the skin of a patient. The sealing mechanism 30 and latch 24 are secure, inexpensive, tamperproof, and easy to apply, yet also capable of nearly instant removal in emergencies. Electronic features do not use metal in the chamber 12 that would impede use of MRI and CT imaging equipment. When used within the chamber 12, many anti-microbial agents can provide higher-than-expected efficacy as the chamber 12 sustains close proximity between the hub 14 and an anti-microbial agent for extended periods, typically hours and days. The chamber 12 can hold anti-microbial agents in place while keeping them away from harsh contact with patients' skin. This isolation permits use of more powerful germ killers while maintaining a safe and pleasant patient experience.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A system for improving central line hygiene, including a chamber for containing central line hubs such that said chamber provides a barrier to said central line hub and protects said central line hubs from germs.

2. The system of claim 1, wherein said chamber includes two concave halves forming a clamshell design operatively attached through a hinge.

3. The system of claim 2, wherein said hinge is formed by tunnels connected to said concave halves and secured by a hinge pin.

4. The system of claim 2, wherein said hinge is further defined as a living hinge of flexible material.

5. The system of claim 2, wherein said hinge includes a sensor and electronics for detecting and recording opening and closing of said chamber.

6. The system of claim 1, wherein said chamber is made of a material chosen from the group consisting of polyamide, polyoxymethylene, polypropylene, acrylonitrile butadiene styrene, polycarbonate, and UV-transmissive acrylic.

7. The system of claim 1, wherein said chamber further includes a skin operatively attached to a location chosen from the group consisting of an outside of said chamber, an inside of said chamber, and both an outside and an inside of said chamber.

8. The system of claim 7, wherein said skin is made of a material chosen from the group consisting of medical-grade ethylene vinyl acetate, silver impregnated foam, medical-grade or biocompatible silicon rubber, and a transparent thermoplastic capable of elongation.

9. The system of claim 7, wherein said skin includes radiusing and chamfering.

10. The system of claim 7, wherein said skin affects a seal of said chamber.

11. The system of claim 2, wherein said chamber further includes a latch at a side opposite said hinge for sealing said chamber.

12. The system of claim 11, wherein said latch includes a mode chosen from the group consisting of single-use mode, re-closable mode, rapid-release mode, child-resistant mode, and combinations thereof.

13. The system of claim 1, wherein said chamber includes at least one sub-chamber for housing a sensor or burstable material.

14. The system of claim 1, wherein said chamber includes at least one portal through which at least one line of tubing enters said chamber.

15. The system of claim 15, wherein said tubing terminates and said chamber works in terminated mode.

16. The system of claim 15, wherein said tubing exits through a second portal in said chamber to operate in through mode.

17. The system of claim 16, wherein said chamber includes a third portal for access.

18. The system of claim 15, wherein said portals are formed by opposing semi-circles set into each half of said concave halves.

19. The system of claim 18, wherein said semi-circles include a sealing mechanism to seal said tubing.

20. The system of claim 19, wherein said sealing mechanism includes tethered plugs for sealing without tubing.

21. The system of claim 19, wherein said sealing mechanism is made of a low-durometer silicon rubber material.

22. The system of claim 1, wherein said chamber further includes a composition chosen from the group consisting of desiccants, anti-microbial agents, and combinations thereof.

23. The system of claim 1, wherein said chamber includes an observation aperture.

24. The system of claim 1, wherein said chamber includes a leak-responsive coating that provides a change chosen from the group consisting of color, shape, or combinations thereof in response to contact with fluid.

25. The system of claim 1, wherein said chamber includes a burstable material that is released into said chamber upon closure.

26. The system of claim 1, wherein said system further includes a wand for irradiating said chamber and measuring parameters of said chamber.

27. The system of claim 26, wherein said wand operates by automatically recognizing said chamber.

28. The system of claim 26, wherein said wand includes an array of UV LEDs.

29. The system of claim 26, wherein said chamber includes a pigmented region that fluoresces upon exposure to said wand.

30. A kit for a system for improving central line hygiene comprising:
a chamber for containing central line hubs such that said chamber provides a barrier to said central line hub and protects said central line hubs from germs;
a wand for irradiating said chamber and measuring parameters of said chamber; and
instructions for use.

31. A method of protecting a central line hub from germs, including the steps of:
enclosing the central line hub with a chamber; and
preventing germs from entering the chamber.

32. The method of claim 31, wherein the chamber includes two concave halves forming a clamshell design operatively attached through a hinge and a latch at a side opposite the hinge.

33. The method of claim 32, wherein said enclosing step further includes the step of sealing the chamber from germs through a hinge and latch operatively connected to the chamber.

34. The method of claim 31, wherein the chamber includes a skin, and wherein said enclosing step further includes the step of sealing the chamber from germs through the skin.

35. The method of claim 31, further including the step of opening the chamber by releasing the latch.

36. The method of claim 31, further including the step of collecting data regarding opening and closing of the chamber.

37. The method of claim 31, further including the step of checking for leaks in the chamber through an observation aperture.

38. The method of claim 31, further including the step of irradiating the chamber with UV light from a wand and removing germs inside the chamber, and the chamber fluorescing once exposed to the wand.

39. The method of claim 31, further including the step of measuring parameters of central line maintenance events with the wand.
